(19) United States
(12) Patent Application Publication
(10) Pub. No.: US 2014/0052080 A1
(43) Pub. Date: Feb. 20, 2014

(54) Disposable and portable garment for treating hands or feet

(71) Applicants: Lois Gibson, Bradley Beach, NJ (US); Carol L. Heindl, Asbury Park, NJ (US)

(72) Inventors: Lois Gibson, Bradley Beach, NJ (US); Carol L. Heindl, Asbury Park, NJ (US)

(21) Appl. No.: 13/573,019

(22) Filed: Aug. 15, 2012

(51) Int. Cl. A61M 35/00

(52) U.S. Cl. CPC A61M 35/00
USPC 604/290; 604/292; 604/293

(57) ABSTRACT

The present invention is a disposable garment, such as a glove, a sock, or a shoe, for treating foot or hand inflammation or disease. In a preferred embodiment, the garment comprises a flexible outer cover that is nonporous and an inner layer closely adhered to the inside of the outer cover. The outer cover may preferably have a flexible closure at the level of the ankle or wrist, arranged such that liquids will be retained therein. The inner layer serves as a reservoir for a selected therapeutic product, and preferably comprises a bladder. Preferably, the bladder has a perforation, and may be disposed within the inner layer in a position allowing a user to apply pressure to bladder with his foot or hand. A therapeutic product in liquid form is stored within the reservoir, and is released upon application of pressure by the user's foot or hand.

### BACKGROUND OF THE INVENTION

1. Field of the Invention

This invention relates to garments, including but not limited to shoes or gloves, for the application of medication or related substance to the foot or hand of a patient in need of such medication or related substance.

2. Description of Related Art

The present invention provides a disposable method to treat tired and achy feet or hands to obtain an anti-inflammatory, analgesic, or soothing effect. The average person takes between 8,000 and 10,000 steps each day. At the end of a day or period of standing around, walking, hiking or even just driving a car for a long time, a person's feet can feel very sore and tired. Hands can suffer the same effects as feet due to repetitive tasks such as computer use or household chores. Any activity which requires one to use their hands excessively and constantly can cause sore hands. Common problems in the fingers, hands, knuckles, thumbs, and wrists may be caused by tiny contraction knots in overworked or traumatized muscles. While these problems with hands and feet are very common, most people do not have the time to rejuvenate their feet or hands.

Soaks can be very soothing and counter the soreness in both hands and feet. Soreness occurs when waste products like lactic acid settle in your muscles through dilated vessels. Room or colder temperatures can constrict vessels, limiting the amount of waste product that accumulates, relieving soreness. Soaking in room temperature or colder water can therefore be very effective in relieving soreness.

Calluses are areas of hardened, thick skin that build up over time because of repeated contact and friction, and typically occur on the heels and balls of feet. Corns are painful calluses that occur on small specific areas, usually toes. They are typically caused by shoes that create too much pressure and friction, as in pointed toes shoes. Cracked feet are caused by conditions such as dehydration of the dermis, or obesity, which places an excess amount of pressure on the heels. In addition to treating inflammation of feet and hands, foot and hand soaks are effective at for treating calluses, corns, and dry and cracked, by promoting better circulation and moisturizing the hands and feet.

Garments to aid in the treatment of foot or hand diseases are known in the art. Remedies for foot or hand disease include creams, powders, and lotions, and garments, including gloves or booties containing preparations such as creams, powders, or lotions. For example, U.S. Pat. No. 3,342,182 discloses a packaged cream applicator that is a glove having a soft, absorbent inner side and a substantially impermeable outer side. The inner side or the lining is impregnated with a medical preparation such as a cosmetic cream. The device is sold to the consumer ready for use. The user dampens or moistens her hands and inserts them into the gloves and simply wears the impregnated gloves. By manipulating the hands and the fingers during ordinary activities, the packaged cream is mixed with the moisture in the skin and is dispensed evenly on the skin. The problem with this glove is that there is uneven distribution of the cream to the skin. Further, the glove is provided with the inner absorbent side impregnated with medical preparation, rendering it difficult to transport. U.S. Pat. No. 4,122,554 and U.S. Pat. No. 4,185,330 teach a disposable cosmetic glove which has a first and a second layers of lotion impervious material and an inner glove lining formed by first and second lotion absorbent layers. The absorbent layers are uniformly impregnated with a cosmetic lotion. Alternatively, the user could apply the lotion to his or her hands first and then put the gloves on to saturate the inner lining prior to use. The shell and lining layers are sealed together along the marginal edge of the glove and a small amount of thermosetting adhesive is deposited at a number of selected locations around the marginal edge of the glove to anchor the liner as the glove is worn. U.S. Pat. No. 4,567,065 provides dispenser gloves and stockinets which may or may not contain the treating agent. The glove is prepared from vinyl-latex and the treating agent is applied to the lumen of the glove prior to packaging. U.S. Pat. No. 5,614,202 discloses a moisturizing glove with three layers. The exterior layer is a non-porous material, and is formed on the top side of the middle layer. The middle layer is saturated with a lotion. An inner layer includes a plurality of pores formed on the bottom side of the middle layer. The inner layer creates a cavity for receiving and enveloping a human extremity such as a hand. U.S. Pat. No. 2,601,851 discloses a bag-like envelope for the feet specifically for the application of medication to treat ringworm or athlete's foot. The device requires breaking an inner envelope containing the medication by exerting pressure on the foot bag envelope. U.S. Pat. No. 2,916,036 discloses rubber gloves with an elaborate composition of layers or holding areas containing globules therein such that the warmth of the hand to which the gloves are applied may allow a medication to escape down the fibers of the glove to the skin of the user. U.S. Pat. No. 3,116,732 discloses a disposable glove with an outer layer of leak-proof material, an inner layer of porous material and a plurality of reservoirs for containing a hand-treatment medication. U.S. Pat. No. 6,117,119 discloses a gelatinous sock, glove, or other protective garment formed from a blend of thermoplastic material with various vitamins and oils incorporated therein. U.S. Pat. No. 5,526,584 discloses a sock-like shoe insert an upper having waterproof layer with a sole having a waterproof, non-porous plastic film that can be stretched in two directions. U.S. Pat. No. 5,682,617 discloses a latex stocking bandage that is non-porous and is used to maintain treatment modalities, such as creams and ointments, in contact with the skin of the user. U.S. Pat. App. 2003/0056276 discloses a manicure/pedicure glove for application of dermal treatments to the hand or foot. The glove has two layers, with a breathable outer layer surrounding in inner layer impregnated with a wax-based treatment. U.S. Pat. App. 2003/0130216 discloses a disposable sock-like garment with a nonporous outer layer, not having a closure at the ankle is sufficient to retain fluids, but having a hydrating structure as an inner layer. U.S. Pat. No. 4,622,035 discloses a boot with an impervious outer covering and with a medicament supply network which provides a continuous supply of medicament to an absorbent lining around the inside of the outer covering. The medicament supply network contains a medicament reservoir located adjacent the opening of the boot and a series of supply fingers which extend along the inside of the contours of the outer covering to the extremities of the boot. U.S. Pat. No. 4,186,445 discloses a disposable cosmetic glove which includes an outer glove shell of lotion impervious material and an inner glove lining of lotion absorbent material, with the inner glove lining preferably uniformly impregnated with a cosmetic lotion

In view of the foregoing, it should be apparent that there still exists a need in the art for an apparatus which can be readily used to apply a lubricating, moisturizing or medicinal type agent to the hand or foot in a quick, clean and efficient manner and a method of manufacturing same. There is specifically a need for such an apparatus that cleanly and efficiently applies liquid content including the agent to be applied, without allowing for leakage of the liquid content. Thus, an objective is to provide a disposable therapeutic apparatus for delivering a lubricating, moisturizing or medicinal type agent in liquid form to the hand or foot without leakage issues. Another objective is to provide apparatus which will deliver a lubricating, moisturizing or medicinal type agent to the hand or other part of the body for an extended period of time. Another objective is to provide apparatus that will deliver such agents in liquid form in such a manner as to permit the user to wear an additional garment, such as a shoe or sock, over the outer surface of the apparatus. Another objective is to provide an efficient and inexpensive means for applying lubricating, moisturizing or medicinal type agent in liquid form to the foot or hand of the user in a simple manner. Another objective is to provide a method of making a disposable hand or foot treatment apparatus meeting the aforementioned objectives.

### BRIEF SUMMARY OF THE INVENTION

The present invention provides a disposable garment with an absorbent inner layer and a nonporous outer layer, with the garment designed to allow for the soaking of the user's hands or feet while the person is mobile covering designed to soak hands or feet while a person is mobile.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 presents a diagrammatic side view of a sock-type embodiment of the present invention with the bladder intact and filled with a suitable fluid.

FIG. 2 presents a diagrammatic side view of a sock-type embodiment of the present invention with the bladder not intact and the fluid situated in the sock.

FIG. 3 presents a diagrammatic side view of a glove-type embodiment of the present invention with the bladder intact and filled with a suitable fluid.

FIG. 4 presents a diagrammatic view of a glove-type embodiment of the present invention with the bladder not intact and the fluid situated in the sock.

### DETAILED DESCRIPTION OF THE INVENTION

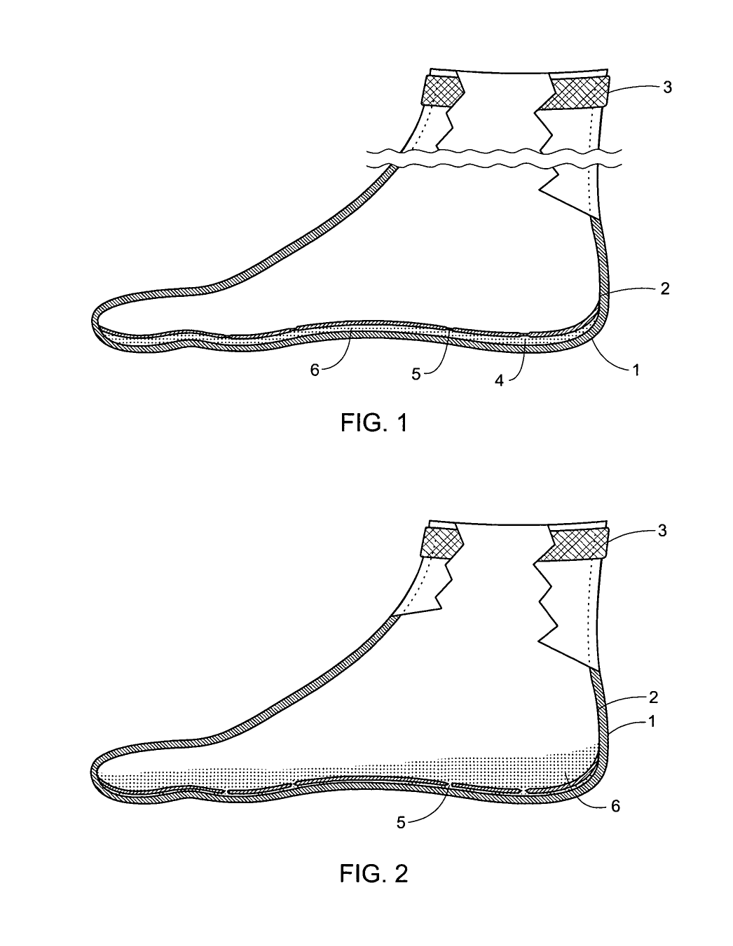

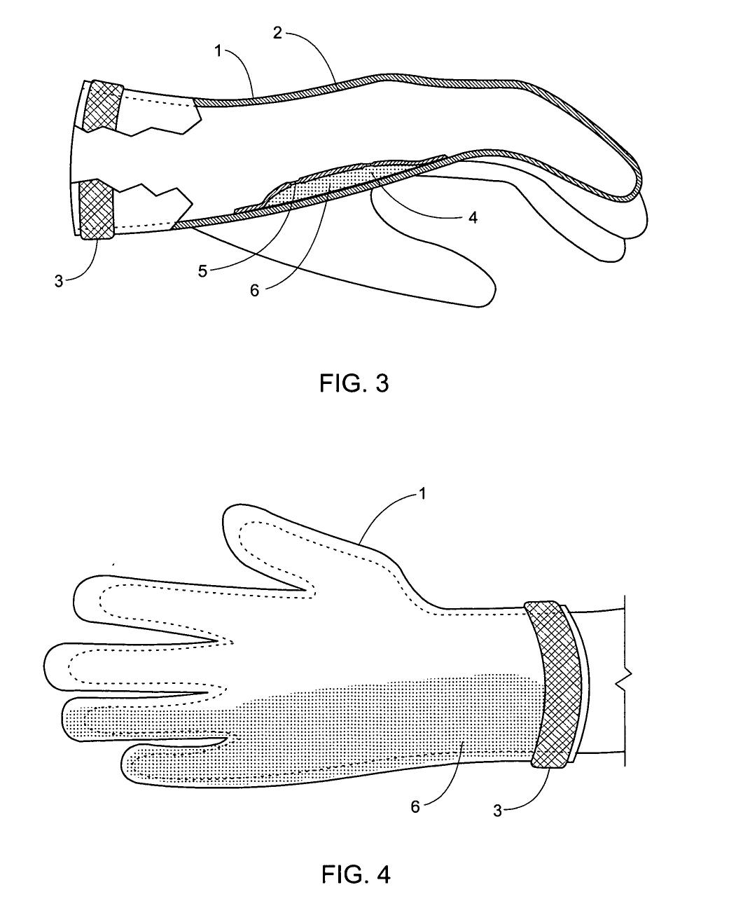

The present invention is a disposable garment, such as a glove, a sock, or a shoe, for treating foot or hand inflammation or disease. An embodiment of the present invention will be described by way of example and reference to the accompanying drawings. In a preferred embodiment, an example of which is illustrated in FIG. 1, the garment comprises a flexible outer cover 1 that is nonporous and an inner layer 2 closely adhered to the inside of the outer cover. The outer cover may preferably have a flexible closure 3 at the level of the ankle or wrist, arranged such that liquids will be retained therein. The inner layer serves as a reservoir for a selected therapeutic product, and preferably comprises a bladder 4. Preferably, the bladder has at least one perforation 5, and may be disposed within the inner layer in a position allowing a user to apply pressure to bladder with his foot or hand. A therapeutic product in liquid form 6 is stored within the reservoir, and is released upon application of pressure by the user's foot or hand. In FIG. 1, a sock-like embodiment is shown. FIG. 2 illustrates the sock-like embodiment after the liquid is released from the bladder. FIG. 3 illustrates an example of a glove-like embodiment of the invention, and FIG. 4 illustrates the glove-like embodiment after the fluid has been released from the bladder.

The present invention is a disposable system and therapeutic device for relieving pain and stress in a hand or foot. The bladder serves as a receptacle for fluids such as water and certain nutraceutical, botanical and United States Food and Drug Administration-designated GRAS (generally recognized as safe) compounds intended to sooth the foot or hand. When pressure is applied to the bladder or absorbent pad, the liquid content is expelled to cover the foot or hand, thereby revitalizing and soothing the foot or hand tissue. The apparatus can be used alone, or may be used with shoes, sneakers, or any type of foot wear or hand covering. Moreover, because the apparatus is designed to prevent the release of the liquid content through or outside the entrance to the garment, the user may go about his day, maintaining his mobility while bathing and revitalizing his feet or hands.

The present invention may preferably comprise an absorbent material or a bladder which contain a mixture of water and or other liquids with FDA designated GRAS substances or mixtures dissolved therein. These GRAS substances may include substances having anti-inflammatory, analgesic, or soothing effect or a combination of all three to revitalize sore and tired feet and hands. These substances may include organic, natural or synthetically-produced substances, which can be dissolved in water to produce the desired effect, either as a single or collective effect. Examples of such substances include, but are not limited to, menthol, lavender, tea tree oil, mints (i.e. peppermint), aloe, glycerin, and helichrysum. By way of example, but not limited to, a combination of the following is designed to produce the desired effects of reduction of inflammation, analgesia and soothing of sore feet and hands (joints, ligaments and muscles): water, wintergreen (with an active component similar to cortisone, a known anti-inflammatory agent), lavender (with a relaxing effect), marjoram (for relief of muscular problems) and helichrysum (providing an anesthetic effect).

The outer covering of the garment may be made of a variety of water proof materials including but not limited to latex, rubber, plastic, and nonlatex substances designed to retain the liquid content and cover the foot and heal up to the ankle and the hand up to the wrist. The covering is preferably will flexible, such as in latex or rubber, allowing the easy insertion of the foot or hand. The garment also preferably has a flexible closure at the level of the ankle or wrist that is sufficiently tight to prevent leakage of the liquid content of the garment. The garment can thus be worn alone or with shoes, slippers or other outer covering for both feet or a glove or mitten-shaped covering for the hands.

In a preferred embodiment, the garment is provided to the user with a premixed liquid solution including the GRAS compound enclosed in the bladder portion.

The presently described apparatus is effective by hydrating the skin with a combination of water and other medically-effective products via contact with the absorbent bladder pad to produce an anti-inflammatory, analgesic and soothing effect. The garment may be worn for a specified period of time. The garment is then disposed of by the user.

The present is also a method of making a garment as herein described. In a preferred embodiment, a disposable foot soak, comprising an outer layer and an inner layer, is made with the inner and outer layers adapted to be towards and away from the foot and molded together. The foot soak is shaped around the foot, so shoes and socks may be worn over the foot soak, with an elastic band around the top of the foot soak and would extend around the ankle preventing leakage of liquid. The outer layer is made of a waterproof substance such as plastic, and is fused onto a perforated or pressure released inner layer (bladder), which contains selected products impregnated into the inner layer or bladder, which is moistened with a liquid substance. The inner layer comprises a top sheet which will retain the fluids in the inner layer until it is pressure activated.

In a preferred embodiment, a foot soak method is constructed by fusing an impervious outer layer made of non-porous, waterproof material (i.e. rubber, plastic or non-latex) with an inner layer. The outer layer is designed and molded as one piece to fit around the foot with an upper opening for receiving a user's foot. The inner layer will have a top sheet which will retain the fluids in the inner layer until it is pressure activated. The inner layer of the disposable foot soak includes a liquid non-pervious top sheet which is released by pressure and it is perforated to allow the liquid contained in the inner layer (core or bladder) to be released and bathe the foot. The outer liner is fused to the bottom of the inner layer which has a top sheet as described. The top sheet is joined to the top of the core layer which faces away from the outer layer by a process which will contain the fluid until it is pressure released by the insertion of a foot into the opening and applying pressure to the top sheet and inner layer releasing the fluid to bath the foot. The most upper part of the outer layer has an elastic band to ensure that none of the fluids leak out of the system. Once the system is placed on the foot or hand of the user, the user can place shoes and socks over the system.

While the invention has been described in connection with specific embodiments thereof and in specific uses, various modifications thereof will occur to those skilled in the art without departing from the spirit and scope of the invention as set forth in the appended claims. Moreover, the terms “consisting”, “comprising” and other derivatives from the term “comprise” are intended to be open-ended terms that specify the presence of any stated features, elements, steps, or components, and are not intended to preclude

What is claimed is:

1. A disposable garment that comprises:
(1) a flexible outer cover that is nonporous, having a flexible closure at the level of the ankle or wrist, arranged such that liquids will be retained therein,
(2) an inner layer closely adhered to the inside of the outer cover, said inner layer serving as a reservoir for therapeutic product, and said inner layer comprising a bladder,
wherein said bladder has a perforation, and wherein said bladder is disposed within such inner layer in a position allowing a user to apply pressure to said bladder with user's foot or hand, and
(3) a therapeutic product in liquid form stored within the reservoir, wherein said therapeutic product is released upon application of pressure by the user's foot or hand.

2. The garment of claim 1, comprised of a shoe.

3. The garment of claim 1, comprised of a sock.

4. The garment of claim 1, comprised of a glove.

5. A method of treating at least one of inflamed feet, inflamed hands, calluses, or cracks, comprising providing a garment in accordance with claim 1 to a user suffering from at least one of such conditions.

6. A method of making a garment according to claim 1, comprising
(1) forming an inner layer by joining a top sheet having at least one perforation to a bottom sheet, forming a bladder between said top and bottom sheet,
(2) fusing an impervious outer layer made of non-porous, waterproof material with said inner layer, and
(3) molding the fused outer layer and inner layer as one piece to fit around the foot or hand with an upper opening for receiving a user's foot or hand.
